(19) United States
(12) Patent Application Publication
(10) Pub. No.: US 2005/0114918 A1
(43) Pub. Date: May 26, 2005

(54) System and method of embryo delivery for manufactured seeds

(76) Inventors: Edwin Hirahara, Federal Way, WA (US); Paul G. Gaddis, Seattle, WA (US)

Correspondence Address: WEYERHAEUSER COMPANY;INTELLECTUAL PROPERTY DEPT., CH 1J27, P.O. BOX 9777, FEDERAL WAY, WA, 98063, US

(21) Appl. No.: 10/982,951

(22) Filed: Nov. 4, 2004

Related U.S. Application Data
(60) Provisional application No. 60/525,449, filed on Nov. 25, 2003.

(51) Int. Cl. A01H 1/00; C12N 15/82; G06K 9/00

(52) U.S. Cl. 800/278; 382/128

(57) ABSTRACT

A embryo delivery system 20 is composed of an embryo orientation assembly 22, a transfer assembly 24, and an embryo reception assembly 26. In operation, the embryo delivery system 20 retrieves plant embryos one at a time from a position on the manufactured seed production line with microtweezers and places each embryo into a separate growing medium, such as a seed coat. The embryo delivery system 20 further includes a control system 28 having a computer 56 or other general computing device for automating the embryo delivery process.

### CROSS REFERENCE TO RELATED APPLICATION

The present application claims the benefit of U.S. Provisional Application No. 60/525,449, filed Nov. 25, 2004, under 35 USC §119(e).

### FIELD OF THE INVENTION

The present invention relates generally to manufactured seeds and, more particularly, to a system and method for the delivery of plant embryos to a manufactured seed coat.

### BACKGROUND OF THE INVENTION

Modern agriculture, including silviculture, often requires the planting of large numbers of substantially identical plants genetically tailored to grow optimally in a particular locale or to possess certain other desirable traits. Production of new plants by sexual reproduction can be slow and is often subject to genetic recombinational events resulting in variable traits in its progeny. As a result, asexual propagation has been shown for some species to yield large numbers of genetically identical embryos, each having the capacity to develop into a normal plant. Such embryos must usually be further cultured under laboratory conditions until they reach an autotrophic “seedling” state characterized by an ability to produce their own food via photosynthesis, resist desiccation, produce roots able to penetrate soil and fend off soil microorganisms.

Some researchers have experimented with the production of artificial seeds, known as manufactured seeds, in which individual plant somatic or zygotic embryos are encapsulated in a seed coat, such as those disclosed in U.S. Pat. No. 5,701,699, issued to Carlson et al., the disclosure of which is hereby expressly incorporated by reference.

Typical manufactured seeds include a seed coat, a synthetic gametophyte and a plant embryo. Typically, the seed coat is a capsule having a closed end and an open end. The synthetic gametophyte is placed within the seed coat, such that the gametophyte substantially fills the seed coat. A cotyledon restraint may be centrally located within the synthetic gametophyte. The cotyledon restraint includes a centrally located cavity extending partially through the length of the cotyledon restraint and sized to receive the plant embryo therein. The well-known plant embryo is approximately 4-7 millimeters in length and roughly 0.5 millimeters in diameter. The shape of the plant embryo is somewhat cylindrical, but is irregular in cross-section and varies in diameter along its length. The plant embryo includes a radicle end and a cotyledon end. The plant embryo is deposited within the cavity of the cotyledon restraint cotyledon end first. The plant embryo is typically sealed within the seed coat by at least one end seal.

In the past, delivery of the plant embryo within the seed coat has utilized either conventional manually operated tweezers or vacuum pick-up devices to transfer the plant embryo through the manufactured seed production line. In such transfer systems that utilize conventional tweezers, the plant embryos are placed manually in separate seed coats, one at a time, by technicians. In such transfer systems that utilize vacuum pick-up devices, the plant embryos one at a time are grasped at their sides from a first position and transferred to a second position by an automated robotic arm. Attached to the end of the robotic arm is a pick-up head to which a source of vacuum to connected. The pick-up head includes a tip having a tip opening designed to grasp and hold a single plant embryo via vacuum pressure. After the pick-up head grasps the embryo, the embryo is positioned to acquire its morphological measurements and the location measurements for the radicle end. Then, the embryo is repositioned so that the embryo is held at the radicle end of the embryo, and is subsequently transferred to the second position for placing the embryo into the seed coat. Once the robotic arm is moved to the second position, the source of vacuum is shut off to release the embryo.

Although such plant embryo delivery systems are effective at transporting plant embryos, they are not without their problems. For example, when using conventional manually operated tweezers, the amount of force applied to the embryos is difficult to control. This results in the possibility of damaging the embryos, and the implementation of force sensors for such a small object using conventional methods to overcome this deficiency is too impractical for commercial success. When using vacuum pick-up heads, the embryo is not always successfully grasped due to the random orientation of the embryos and the variability of the size and shapes of the embryos. Additionally, the embryo surface is curved, which can prevent an adequate seal with the pick-up head tip opening. Such an imperfect seal may allow sufficient air flow around the embryo, resulting in a deficient vacuum to form. Accordingly, a lack of suction force is present to grasp and hold the embryo during the transfer process, which leads to unsuccessful transfers. Unsuccessful transfers of viable embryos are costly in modern automated material handling systems.

Secondly, with both aforementioned transfer methods, a problem may exist when either the operator or the automated pick-up head attempts to release the embryo into the seed coat. Specifically, since the embryos are kept moist or wet to prevent damage from desiccation, the embryo may remain attached to the tip of either the tweezers or the pick-up head due to the surface tension formed between the moisture on the embryo and the contact area of the tweezers or the pick-up head tip. In the case of conventional tweezers, to release the embryo, the technician typically positions the embryo to contact the side of the cotyledon restraint opening to create surface tension therebetween to overcome the surface tension associated with the tweezer tips. In the case of the vacuum pick-up head, a puff of air pressure is expelled out of the tip opening to overcome the surface tension and to force the embryo out of the vacuum head. In some instances, the burst of air flow is either insufficient to release the embryo or too great, in which case, the embryo is damaged by the impact force of the embryo against the bottom of the restraint. In either case, viable embryos may be wasted, which is costly in commercial applications. Further, the effects of surface tension and the conventional methods for overcoming the same may cause unwanted movement of the embryo, which in turn, affects the orientation of the embryo for insertion into the seed coat, and may lead to improper placement of or damage to the embryo.

### SUMMARY OF THE INVENTION

The present invention is directed to an embryo delivery system that addresses the deficiencies of the prior art and others by employing automated microtweezers in embryo transfer process. The microtweezers, as will be described in detail below, are specifically designed to reduce the contact area of the tweezer tips on the embryos for reducing the surface tension therebetween. The reduction in surface tension results in improved embryo release capabilities for the embryo delivery system.

In accordance with one embodiment of the present invention, a method is provided for delivering embryos. The method includes positioning at least one embryo located on a support surface in a retrieval position. The oriented embryo is retrieved with automated microtweezers by actuation of the microtweezers to a closed position. The microtweezers are movable between a retrievable position and a release position. The automated microtweezers are moved to the release position where a seed coat is positioned relative to the release position. The embryo is then released into the seed coat by actuation of the microtweezers to an open position.

In accordance with another embodiment of the present invention, a method is provided for delivering plant embryos to a growing medium. The method includes imaging a plurality of plant embryos supported on a first surface for obtaining at least one selected plant embryo attribute, and orienting one plant embryo in a predetermined retrieval position based on the plant embryo attribute. The oriented embryo is transferred with microtweezers from the retrieval position to a release position, and then released from the microtweezers into the growing medium at the release position.

In yet another embodiment of the present invention, a method for delivering cultivated embryos is providing in a material handling system having an first positioning table, a transfer device having microtweezers, and a second positioning table. The method includes positioning a surface having a plurality of randomly oriented embryos onto the first positioning table, and obtaining at least one attribute of the randomly oriented embryos. One of the plurality of embryos is then orientated according to the obtained attribute by controlled actuation of the first positioning table so that the embryo achieves a selected, repeatable retrieval position. The embryo is transferred from the surface with the automated microtweezers to a selected, repeatable release position spaced from the surface, and placed into a seed coat positionally controlled by the second positioning table.

### BRIEF DESCRIPTION OF THE DRAWINGS

The foregoing aspects and many of the attendant advantages of this invention will become more readily appreciated by reference to the following detailed description, when taken in conjunction with the accompanying drawings, wherein:

FIG. 1 is one embodiment of an embryo delivery system constructed in accordance with the present invention;

FIG. 2 is an alternative embodiment of the embryo delivery system constructed in accordance with the present invention;

FIG. 3 is a partial perspective view of the microtweezers retrieving a qualified embryo;

FIG. 4 is a partial side view of the reception assembly, wherein the qualified embryo is released from the microtweezers and placed within a growing medium, such as a manufactured seed; and

FIG. 5 is a block diagram depicting the components of the embryo delivery systems of FIGS. 1 and 2.

### DETAILED DESCRIPTION OF THE PREFERRED EMBODIMENTS

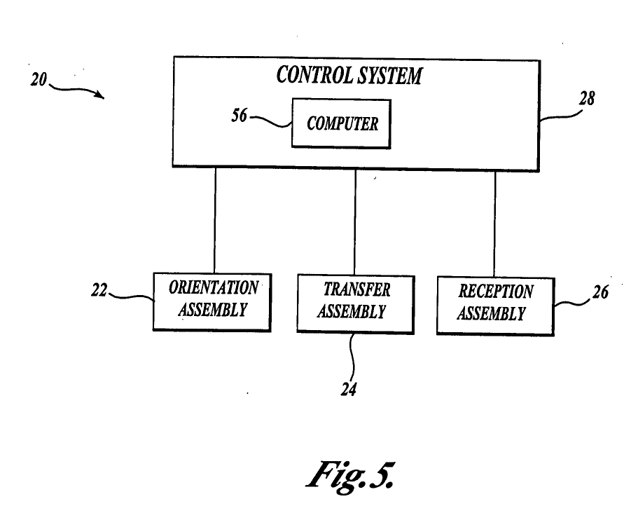

The present invention will now be described with reference to the figures where like numerals represent like elements. FIG. 5 is a block diagram illustrating one embodiment of an embryo delivery system 20 constructed in accordance with the present invention. The embryo delivery system 20 is composed of an embryo orientation assembly 22, a transfer assembly 24, and an embryo reception assembly 26. In operation, the embryo delivery system 20 retrieves plant embryos one at a time from a position on the manufactured seed production line and places each embryo into a separate growing medium, such as a seed coat. To this end, the orientation assembly 22 orients the plant embryos to be grasped by the transfer assembly 24. The transfer assembly 24 sequentially grasps the embryos from the orientation assembly 22 and moves the embryos to a second location where the embryos are received by the embryo reception assembly 26. The embryo delivery system 20 further includes a control system 28 having a computer 56 or other general computing device. The control system 28 sends and receives control signals to and from the assemblies 22, 24, and 26 for automating the embryo delivery process.

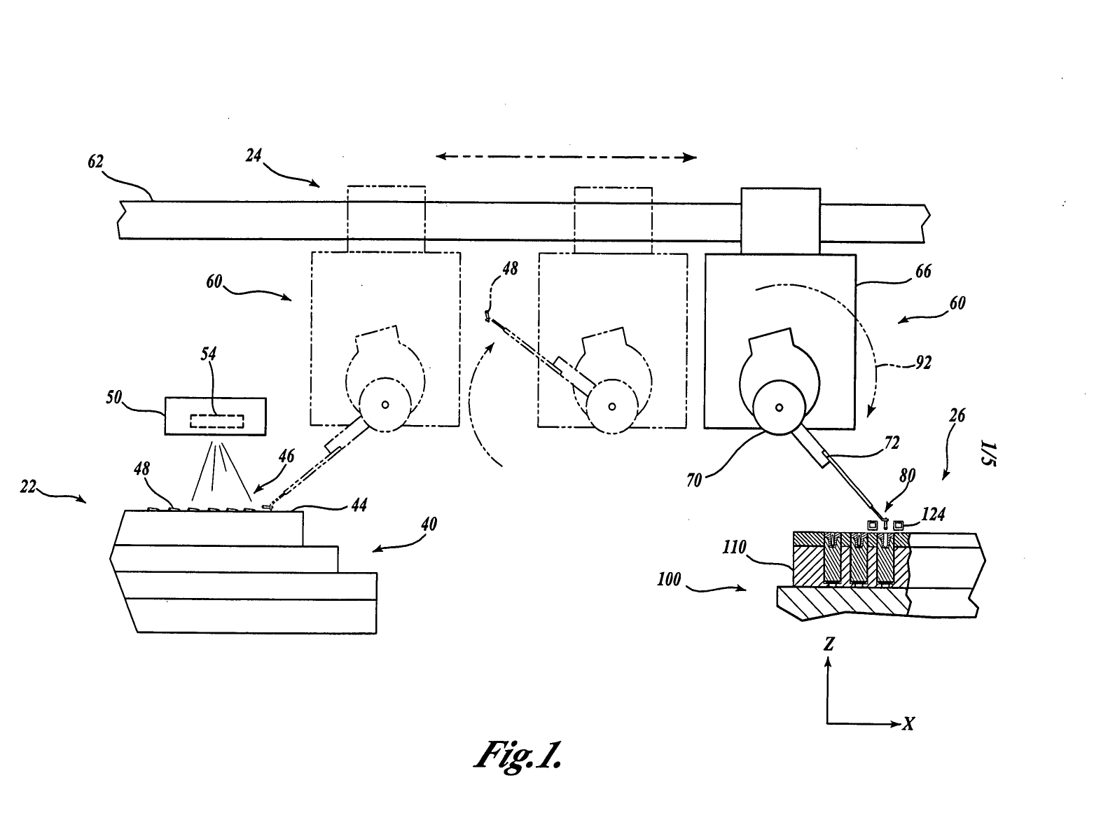

Referring now to FIG. 1, the embryo orientation assembly 22 will now be described in greater detail. As may be seen by referring to FIG. 1, the orientation assembly 22 includes a precision X-Y-rotation positioning table 40. The positioning table 40 selectively translates in two dimensions, and rotates about an axis orthogonal to the translating directions. In particular, the positioning table 40 is permitted to move fore and aft along the X direction, side-to-side along the Y direction, as well as rotating about the Z-axis for affecting angular displacement. In one embodiment of the present invention, the positioning table 40 may be conventionally assembled from two linear motion tables, one for the X direction and one of the Y direction, such as Model F55-332, and one rotary motion table, such as Model F55-327, all of which are commercially available from Edmund Industrial Optics, Barrington, N.J. Located on top of the positioning table 40 is a support surface 44, such as a Petri dish, on which a plurality of embryos 46 are randomly oriented. The embryos 46 may be randomly placed on the support surface 44 manually by technicians or by an automated process from the manufactured seed production line.

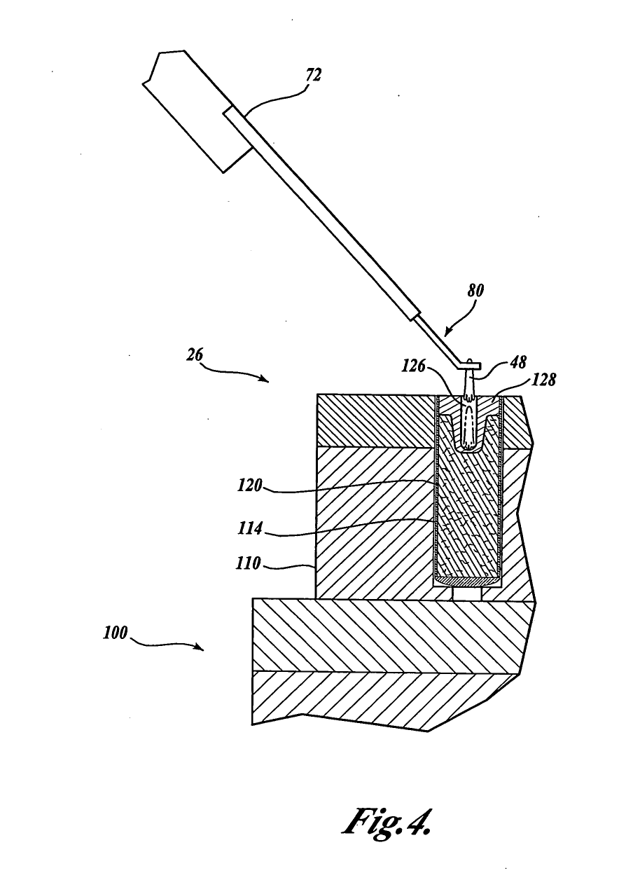

The orientation assembly 22 further includes an imaging system 50 or other suitable system for obtaining attributes of the plant embryos 46. The imaging system 50 may obtain any number of plant embryo attributes, such as size, shape, axial symmetry, cotyledon shape or development, surface texture, color, etc. In one embodiment, the imaging system 50 obtains either size or size and shape measurements, and based on these measurements, the embryos 46 will be classified as unqualified or qualified plant embryos. To be classified as a qualified embryo, the measurements of the embryo should indicate, within a sufficient tolerance, that the embryo will fit into the opening 126 of a cotyledon restraint 128 (See FIG. 4). It has been determined by the inventors of the present invention that such a selection criteria will yield an acceptable percentage of viable embryos.

The aforementioned attributes are obtained by the imaging system 50 by first acquiring and then digitally storing, if necessary, images of the plant embryos 46 by a well known digital imaging camera 54. The acquired and digitally stored images are then processed by a software program executed by the computer 56 of the control system 28 (See FIG. 5). The software program makes a qualitative determination of each plant embryo 46, and based on predetermined parameters, size and shape in this case, defines stores which plant embryos are qualified, now referred to as qualified embryos 48. In addition to processing the images taken by the digital imaging camera 54 for selected embryo attributes, the software program also determines external embryo attributes, in this case, positional information associated with each discrete qualified plant embryo 48. Since each growing medium is to receive a single qualified embryo, it will be appreciated that a selection criteria, including either size or shape and size, will disqualify groups or clusters of embryos that may be present on the support surface 44.

In an alternative embodiment, the plant embryos 46 may be qualified or otherwise determined to be suitable for germination based on other criteria, for example, surface texture, color, IR absorption or reflection, Beta ray absorption, axial symmetry, and cotyledon development or any other attribute generally measurable by camera-like sensing devices. To this end, the acquired and digitally stored images of the digital imaging camera 54 may be sent to the computer 56 of the control system 28 (See FIG. 5) and may be processed by a classification software program, such as that disclosed in PCT Application Serial No. PCT/US99/12128, entitled: Method for Classification of Somatic Embryos, filed Jun. 1, 1999, the disclosure of which is hereby incorporated by reference. The software program makes a qualitative determination of the plant embryos, and based on predetermined parameters, defines and stores which plant embryos are qualified.

It will be appreciated that other classification methods and systems may be practiced with the present invention for selecting qualified embryos. For example, the embryos may be classified by the multi-stage screening process disclosed in copending U.S. patent application Ser. No. 10/611,756, entitled: Automated System And Method for Harvesting and Multi-Stage Screening of Plant Embryos, filed Jun. 30, 2003, the disclosure of which is hereby incorporated by reference. Additionally, the embryos may be classified as qualified using a spectroscopic analysis method, such as IR spectroscopy, NIR spectroscopy, or Raman spectroscopy, as disclosed in PCT Application Serial No. PCT/US99/12128, entitled: Method for Classification of Somatic Embryos, filed Jun. 1, 1999. These classification methods may be applied to any absorption, transmittance, or reflectance spectra of the embryos to classify the embryos according to their chemical composition. Other methods using Raman spectroscopy for classifying embryos that may be practiced with the present invention are disclosed in copending U.S. patent application Ser. No. 10/611,530, entitled: Method For Classifying Plant Embryos Using Raman Spectroscopy, filed Jun. 30, 2003, the disclosure of which is hereby incorporated by reference. Further, the apical dome located at the cotyledon end of a plant embryo may be three dimensionally imaged and analyzed for classifying embryos as qualified. Some methods of three-dimensionally imaging an apical dome of a plant embryo can be found in copending U.S. patent application Ser. No. 10/611,529, entitled: Method and System For Three-Dimensionally Imaging an Apical Dome of a Plant, filed Jun. 30, 2003, which is hereby incorporated by reference.

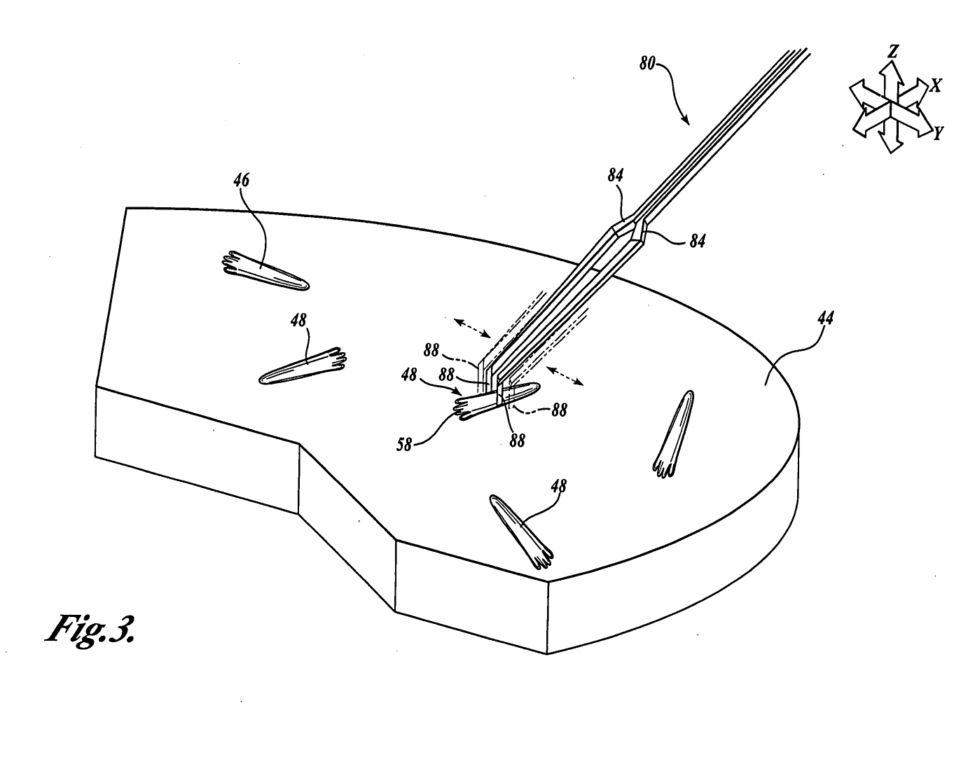

In operation, once a plurality of embryos 46 are randomly positioned on the support surface 44, the imaging camera 54 of the imaging system 50 acquires images of the embryos 46 and transmits the images to the computer 56 (See FIG. 5) for processing. Once a determination is made on each embryo 46 as to whether they are qualified embryos 48 or unqualified embryos, the positional information of each qualified embryo 48 is determined by the computer 56. Next, based on the positional information determined for each qualified embryo 48, the qualified embryo 48 is specifically oriented one at a time by movement of the positioning table 40 to a known retrieval position for retrieval by the transfer system 24. The qualified embryo 48 is then retrieved by the transfer assembly 24, and subsequently transferred to the reception assembly 26, as will be described in detail below. In the embodiment shown, the qualified embryos 48 are sequentially orientated at the retrieval position so that each qualified embryo 48 may be grasped with its cotyledon end 58 aligned in the X direction, as best shown in FIG. 3, facing opposite the reception assembly 26 (facing left of the page in FIG. 1).

In accordance with one aspect of the present invention, the queuing order in which the qualified embryos 48 are selected for retrieval may be specifically determined for improving the throughput of the embryo delivery process. The retrieval order of the qualified embryos 48 from the support surface 44 may be determined by any number of throughput enhancement routines. In the preferred embodiment, the throughput enhancement routine is executed by the computer 56 (See FIG. 5), which sorts the positional information obtained by the imaging system 50 and processed by the computer 56 to select the retrieval order of qualified embryo 48 based on the relative positions of the qualified embryos 48. In operation, the routine first sorts all qualified embryos 48 by rotational position starting with the qualified embryo that has a rotational position, in either degrees or radians, closest to a defined reference position, such as the default positional setting of the position table. Next, the routine controls the positioning table 40 to sequentially orient the qualified embryo 48 to be retrieved by the transfer assembly 24 according to the sorted rotational position information.

Referring now to FIG. 1, the transfer assembly 24 will now be described in greater detail. As was described above, the transfer assembly 24 retrieves a qualified embryo 48 from the support surface 44 at the known retrieval position, and transfers the qualified embryo 48 to a known release position. As may be best seen by referring to FIG. 1, the transfer assembly 24 includes a transfer device 60 selectively movable in a guided manner along a track 62. The selective movement of the transfer device 60 may be effected by any well known linear actuator (not shown), such as a motorized linear screw or a pneumatic piston and cylinder arrangement, and controlled by the control system 28 (See FIG. 5). The transfer device 60 may include a housing 66 having a motorized rotary shaft 70 extending from the housing 66 in the Y direction. The rotary shaft 70 is selectively rotatable between the retrieval position shown in phantom in FIG. 1 (farthest to the left) and the release position, as shown farthest to the right in FIG. 1, and is controlled by the control system 28. Attached to the rotary shaft 70 for rotation therewith is an extension member 72. Attached at the distal end of the extension member 72 are microtweezers 80.

As best shown in FIG. 3, the microtweezers 80 include arms 84 to which microtweezer tips 88 are attached. The tips 88 of the microtweezers 80 are preferably attached to the arms 84 at an angle, for example, 30 degrees, to facilitate the retrieval and release of the qualified embryos 48. The microtweezers 80 may be fabricated out of silicon in an etching or similar process. It will be appreciated that silicon at the contemplated dimensions is capable of flexing. The tips 88 of the microtweezers 80 are movable between an open position shown in phantom in FIG. 3, wherein the space between the tips 88 is sufficient to accept a qualified embryo 48 therebetween, and a closed position, wherein the tips 88 of the microtweezers 80 grasp the qualified embryo 48. The tips 88 of the microtweezers 80 are specifically configured to create a contact surface small enough to minimize the effects of surface tension created by the moisture of the embryo contacting the tips 88 of the microtweezers 80. In particular, the tips 88 are designed with a suitable contact area the allows the release of the qualified embryo 48 when the microtweezers 80 are actuated to the open position, and will minimize the manipulation or movement of the qualified embryo prior to release. In one embodiment, the contact area may be such that when the microtweezers 80 are actuated to release the qualified embryo 48, the weight of the qualified embryo 48 overcomes the surface tension therebetween, which in turn, separates the qualified embryo 48 from the microtweezers 80. In one embodiment, the contact area on each microtweezer tip is approximately 10-100 microns in width, and approximately 2 millimeters in height. It will be appreciated that only a small portion of the 2 mm height will actually contact the embryo, preferably at the distal end, due to the size, shape, and surface curvature of the embryo. Microtweezers that may be practiced by the present invention are commercially available from MEMS Precision Instruments (http://www.memspi.com).

In operation, once the positioning table 40 orients one qualified embryo 48 into the retrieval position, the transfer assembly retrieves the qualified embryo 48. To do so, the transfer device 60 is translated along the track 62 and the microtweezers 80 are rotated by the rotary shaft 70 to the retrieval position, shown in phantom in FIG. 1. The microtweezers 80 may be rotated into the retrieval position contemporaneously with the movement of the transfer device or rotated to the retrieval position subsequent to the movement of the transfer device 60. Once the retrieval position has been achieved, the microtweezers 80 are actuated from the open position, shown in phantom in FIG. 3, to the closed position for grasping the qualified embryo 48. The microtweezers 80 may be actuated to the closed position in a number of different methods; however, in the preferred embodiment, the microtweezers 80 are actuated to the closed position by the application of electrical current to the arms 84 as known in the art, and controlled by the computer. Similarly, the microtweezers 80 may be actuated to the open position, when desired, by shutting off the application of electrical current to the arms 84, as known in the art.

After the qualified embryo 48 is retrieved from the support surface 44, the transfer device 60 is translated along the track 62 to a second, release position, while contemporaneously rotating the shaft 70 in the direction shown by the arrow 92 and opposite of the retrieval direction. Due to the small size of the microtweezers 80 and the qualified embryo 48 to be retrieved, the imaging camera 54 may be operated continuously to provide feedback control information for repositioning the positioning table 40 and/or controlling the actuation of the microtweezers 80 via the computer 86 (See FIG. 5).

While the transfer device 60 is shown linearly translating along the track 62, it will be appreciated that other methods for transferring the qualified embryos from the retrieval position to the release position are possible. For example, the transfer device 60 may employ a robotic swing arm that rotates about the Z-axis for moving the microtweezers between such known positions. Additionally, it will be appreciated that the housing 66 may be a robotic housing capable of movement in the X, Y, and Z directions, as well as rotating about the Z axis. The robotic housing of such a transfer device may be used in conjunction with or in the absence of the positioning table 40 for positioning the microtweezers to retrieve the selected qualified embryos.

Returning to FIG. 1, the reception assembly 26 will now be described in greater detail. As was described above, the reception assembly 26 receives the qualified embryo 48 from the transfer assembly 24 at the release position. As may be best seen by referring to FIG. 1, the reception assembly 26 includes a three-dimensional precision positioning table 100 that selectively translates in three dimensions. In particular, the positioning table 100 is permitted to move fore and aft in the X direction, side-to-side in the Y direction, as well as up and down in the Z direction. In one embodiment of the present invention, the positioning table 100 may be conventionally assembled from two linear motion tables, one for the X direction and one of the Y direction, such as Model F55-332, and one linear motion table for the Z direction, such as Model F53-673, all of which are commercially available from Edmund Industrial Optics, Barrington, N.J.

Located on top of the positioning table 100 is a receptacle tray 110. The receptacle tray 110 includes a plurality of cavities 114 extending vertically therethrough, only one being shown in FIG. 4. As best shown in FIG. 4, received within each cavity 114 is a well known manufactured seed coat 120, such as that disclosed in U.S. Pat. No. 5,701,699, issued to Carlson et al., the disclosure of which is hereby incorporated by reference. The reception assembly 26 further includes at least one position sensor 124 (See FIG. 1), such as a laser micrometer or imaging camera, for obtaining positional information of the qualified embryo 48. The position sensor 124 is located such that the qualified embryo 48 is positioned within the sensor field in the release position. The position sensor 124 determines the location of the center of the cotyledon end 58 (See FIG. 4) of the qualified embryo 48. The positioning table 100 may include an imaging camera (not shown) to precisely locate and store the center of the opening 126 of the cotyledon restraint 128 in the manufactured seed 120. Alternatively, the receptacle tray 110 may be oriented on the positioning table 100 so that the positional information of the restraint opening 126 of each seed coat 120 can be obtained with respect to a known fixed position of the receptacle tray 110 and stored in the control system.

In operation, having the positional information of the cotyledon restraint opening 126 of the manufactured seed coat 120 and the positional information of the cotyledon end 58 of the qualified embryo 48 held by the microtweezers 80 above the positioning table 100, the positioning table 100 precisely adjusts or indexes the location of the receptacle tray 134, such that it moves the opening 126 of the cotyledon restraint 128 to the precise location of the qualified embryo 48 held by the microtweezers 80. At this point, the microtweezers 80 are actuated from the closed position to the open position, and the qualified embryo 48 is released from the microtweezers 80 into the cotyledon restraint 128 of the manufactured seed coat 120.

As was described above in an alternative embodiment, the housing 66 of the transfer device may be a robotic housing capable of movement in the X, Y, and Z directions. The robotic housing of such a transfer device may be used in conjunction with or in the absence of the positioning table 100 for moving the microtweezers into a position to release the qualified embryo into the seed coat.

The operation of the embryo delivery system 20 will now be described by referring to FIGS. 1-5. A plurality of embryos 46 are delivered from the Embryogenesis production line, either manually or by an automated process, and are randomly placed on the support surface 44 of the precision positioning table 40. Next, the imaging camera 54 acquires and digitally stores, if necessary, images that will be used to determine whether any of the embryos 46 can be considered qualified to be placed in a manufactured seed 120.

If the embryos 46 are qualified to be placed in a manufactured seed, the positional information of each qualified embryo 48 is determined and is used to assemble an embryo retrieval queue. In one embodiment of the present invention, the qualified embryos 48 are sorted and arranged in the queue by rotational coordinate information. Once the control system 28 generates a retrieval queue, whether using a throughput enhancement routine or not, the first qualified embryo 48 is oriented by the positioning table 40, through control signals sent by the control system 28, to the precise retrieval position.

Contemporaneously with or sequentially after orientating the qualified embryo 48 to the retrieval position, the control system 28 sends controls signals to the transfer device 60 such that the transfer device 60 translates to the retrieval position and the rotary shaft 70 rotates the microtweezers 80 in the direction opposite the arrow 92 to the embryo retrieval position. Once the microtweezers 80 are in the retrieval position, the microtweezers 80 are actuated to the closed position, thereby grasping the qualified embryo 48 between the microtweezer tips 88. In one embodiment, to improve the accuracy of the retrieval process and to control the force applied to the qualified embryo 48, the imaging system 50 may be continuously acquiring images of the position of the microtweezer tips 88 with respect to the qualified embryo 48, for providing feedback control information to the computer.

After the qualified embryo 48 is retrieved from the support surface 44, the transfer device 60 is translated in the opposite direction along the track 62 to the release position, while contemporaneously rotating the shaft 70 in the opposite direction shown by the arrow 92. In the release position, the microtweezers 80 hold the qualified embryo 48 within a sensor field of the position sensor 124 for obtaining positional information of the cotyledon end 58 of the qualified embryo 48. As best shown in FIGS. 1 and 4, in the release position, the longitudinal axis of the qualified embryo 48 is aligned in the Z direction.

As noted above, simultaneous with or prior to the acquisition of the positional information for the qualified embryo, a second imaging camera associated with the positioning table 100 may locate the position of the opening 126 of the cotyledon restraint 128 in the manufactured seed 120 located on the positioning table 100. Alternatively, the receptacle tray 110 may be oriented on the positioning table so that the positional information of the restraint opening 126 of each seed coat 120 may be obtained and stored by the control system. As a result, having both the positional information of the cotyledon restraint opening 126 of the manufactured seed coat 120 and the positional information of the cotyledon end 58 of the qualified embryo 48, the positioning table 100 then locates itself through control signals sent by the computer 56, to accurately and precisely align the qualified embryo 48 with the opening 126 of the cotyledon restraint 128.

Once the qualified embryo 48 in aligned with the opening 126 of the cotyledon restraint 128, the microtweezers 80 are actuated by the control system 28 to the open position, thereby releasing the qualified embryo 48 into the manufactured seed coat 120. As was described above, the tips 88 of the microtweezers 80 are configured to reduce the contact area against the qualified embryo 48. As such, the weight of the qualified embryo may overcome the surface tension generated between the moist qualified embryo and the contact area of the microtweezer tips 88, thereby releasing the qualified embryo 48 from the microtweezers 80. If for some reason the qualified embryo 48 remains coupled to the microtweezer tips 88, the positioning table 100 may be slightly jogged to release the qualified embryo 48 from the microtweezers 80.

The embodiments of the present invention provide several advantages over currently available embryo delivery systems, some of which will now be explained. First, by employing microtweezers, and controlling its actuation distance, the force exerted on the qualified embryos can be precisely controlled, minimizing potential damage to the qualified embryos. Secondly, by employing the microtweezers, the contact area of the tips of the microtweezers against the embryo is purposefully and significantly reduced as compared to prior art methods, which in turn, minimizes the surface tension forces between the microtweezer tips and the qualified embryo.

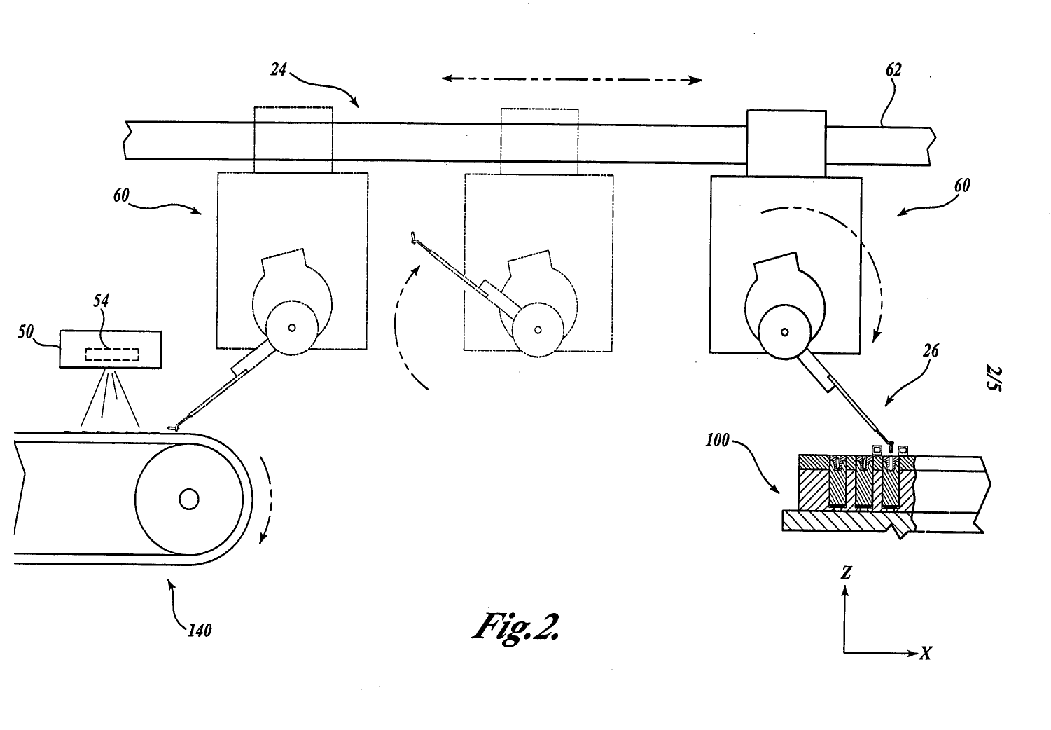

While the orientation assembly 22 in the embodiments shown in FIG. 1 and described herein employ a positioning table, it will be appreciated that other orientation assemblies may be used. For example, as best shown in FIG. 2, the embryos may be retrieved off of a conventional conveyor belt 140. To this end, either the embryos are pre-oriented on the conveyor belt 140 to be grasped by the transfer assembly disclosed herein, or the transfer assembly may employ a multi-directional and rotational robotic housing for orienting the microtweezers with respect the qualified embryos. Additionally, the embryo delivery system 20 may employ the orientation and imaging system disclosed in PCT Application Ser. No. PCT/US00/40720 (WO 01/13702 A2), which is expressly incorporated by reference, for positioning the qualified embryos in a sufficient orientation at the retrieval position. Further, it will be appreciated that the qualified embryo does not have to be directly inserted into the manufactured seed coat at the release position described above. Instead, the qualified embryo may be inserted into a temporary carrier, or could be released onto a different surface in a desired location or orientation. The surface may be a temporary storage location, or a movable surface, such as a conveyor belt, movable web, or positioning table, to name a few.

While the preferred embodiments of the invention has been illustrated and described, it will be appreciated that various changes can be made therein without departing from the spirit and scope of the invention, as claimed.

1. A method for delivering embryos, comprising:
positioning at least one embryo located on a support surface in a retrieval position;
retrieving the oriented embryo with automated microtweezers by actuation of the microtweezers to a closed position, wherein the microtweezers are movable between a retrievable position and a release position;
moving the automated microtweezers to the release position;
positioning a seed coat relative to the release position; and
releasing the embryo into the seed coat by actuation of the microtweezers to an open position.

2. The method of claim 1, wherein the support surface is a conveyor belt.

3. The method of claim 1, wherein the embryo is positioned by a positionally controlled table.

4. The method of claim 3, wherein the positionally controlled table is a precision X-Y-rotation positioning table.

5. The method of claim 1, wherein the embryo is positioned at the retrieval position in a selected orientation.

6. The method of claim 1, further comprising
imaging at least one embryo for obtaining one or more selected embryo attributes.

7. The method of claim 6, wherein the attributes are selected from the group consisting of size, shape, axial symmetry, cotyledon development, surface texture, color, and position.

8. The method of claim 6, wherein positioning the embryo is based on the obtained attribute.

9. The method of claim 1, further comprising
orienting the embryo such that the cotyledon end of the embryo is facing the seed coat in the release position.

10. The method of claim 1, further comprising
obtaining positional information of the embryo at the release position.

11. The method of claim 10, wherein positioning the seed coat relative to the release position is based on the obtained positional information of the embryo.

12. The method of claim 1, wherein positioning the seed coat relative to the release position includes
moving the seed coat relative to the embryo for aligning an opening of the seed coat with an end of the embryo.

13. A method for delivering plant embryos to a growing medium, the method comprising:
imaging a plurality of plant embryos supported on a first surface for obtaining at least one selected plant embryo attribute;
orienting one plant embryo in a predetermined retrieval position based on the plant embryo attribute;
transferring the oriented embryo with microtweezers from the retrieval position and a release position; and
releasing the plant embryo from the microtweezers into the growing medium at the release position.

14. The method of claim 13, wherein the at least one selected embryo attribute is selected from the group consisting of size, shape, axial symmetry, cotyledon development, surface texture, color, and position.

15. The method of claim 13, wherein orienting the embryo includes
obtaining positional information associated with the embryo; and
orienting the embryo based on the obtained positional information.

16. The method of claim 13, wherein the release and retrieval positions are known, repeatable positions.

17. The method of claim 13, further including
calculating size and shape measurements of the embryo based on the obtained image.

18. In a material handling system having an first positioning table, a transfer device having microtweezers, and a second positioning table, a method for delivering cultivated embryos comprising:
positioning a surface having a plurality of randomly oriented embryos onto the first positioning table;
obtaining at least one attribute of the randomly oriented embryos;
orienting one of the plurality of embryos according to the obtained attribute by controlled actuation of the first positioning table so that the embryo achieves a selected, repeatable retrieval position;
transferring the embryo from the surface with the automated microtweezers to a selected, repeatable release position spaced from the surface; and
placing the embryo into a seed coat positionally controlled by the second positioning table.

19. The method of claim 18, wherein obtaining attributes includes
imaging the plurality of randomly oriented embryos; and
calculating the size of the embryos.

20. The method of claim 18, further including
obtaining positional information of the embryo prior to placing the embryo into the seed coat, wherein the seed coat is positioned by the second positioning table based on the obtained positional information.
